(19) United States
(12) Patent Application Publication
(10) Pub. No.: US 2009/0270440 A1
(43) Pub. Date: Oct. 29, 2009

(54) BIOAVAILABILITY OF ACTIVE SUBSTANCES HAVING AN AMIDINE FUNCTION IN MEDICAMENTS

(75) Inventors: Bernd Clement, Kiel (DE); Christiane Reeh, Hamburg (DE)

Correspondence Address: DARBY & DARBY P.C., P.O. BOX 770, Church Street Station, New York, NY, 10008-0770, US

(73) Assignee: Drite Patentportfolio Beteilligungsgesellschaft mbH & Co. KG, Schonefeld/waltersdorf (DE)

(21) Appl. No.: 12/374,300

(22) PCT Filed: Jul. 10, 2007
(86) PCT No.: PCT/DE2007/001216

§ 371 Date: Jul. 14, 2009

(30) Foreign Application Priority Data
Jul. 21, 2006 (DE) 10 2006 034 256.9

(51) Int. Cl. A61K 31/435; C07D 271/06; C07D 471/04; A61K 31/4245; A61P 31/04

(52) U.S. Cl. 514/290; 548/131; 546/98; 514/383

(57) ABSTRACT

The invention relates to the use of N,N′-dihydroxyamidine (I), N,N′-dihydroxyamidine ether (II), N,N′-dihydroxyamidine diether (III), N,N′-dihydroxyamidine ester (IV), N,N′-dihydroxyamidine diester (V) or 4-hydroxy-1,2,4-oxadiazoline (VI) of the formulae cited above, wherein R represents hydrogen, an alkyl and/or aryl radical, as a substitute for an amidine function of a medicament for improving the bioavailability of the medicament.

The present invention relates to the improvement of the bioavailability of medicinal substances which have at least one amidine function and to medicaments comprising correspondingly modified medicinal substances.

Pharmaceutical preparations which comprise an active ingredient having one or more amidine functions show virtually no pharmacological effect on oral use. The precondition for a therapeutic effect of an active ingredient after oral administration is uptake thereof from the gastrointestinal tract. The most important mechanism of such an effect is passive diffusion. The degree of absorption by the passive diffusion route is dependent on the lipophilicity and thus also dependent on the acidity and basicity of the active ingredient.

A highly basic compound such as benzamidine is virtually completely ionized in the stomach (pH 1) and in the small bowel (pH 6.4). Absorption after oral administration, which requires passage through the lipid bilayers of the membranes of the gastrointestinal tract, therefore takes place to only a very small extent. It is to be presumed that all active ingredients having an amidine as functional group will show inadequate absorption on oral use.

The N-hydroxylated derivatives such as the amide oximes show a lower basicity through the introduction of the oxygen atom. Amide oximes are not protonated under physiological conditions. Benzamide oxime represents a model compound for many medicinal substances comprising an amide oxime function [Clement, B. (2002) Drug Met. Rev. 34 565-579]. In the case of ximelagatran it was possible to increase the oral bioavailability by introducing the amide oxime function by comparison with melagatran only from 7% to 14%, however [Clement, B.; Lopian, K. (2003) Drug Met. Dispos. 31 645-651]. There is thus still an urgent need for medicinal substances having an amidine function which are efficiently absorbed via the gastrointestinal tract after oral administration.

It is therefore the object of the present invention to increase the oral bioavailability of substances which comprise an amidine function.

The object is achieved according to the invention by the features of claim 1. The dependent claims represent advantageous refinements of the invention.

According to the use according to the invention, replacement of at least one amidine function by N,N′-dihydroxyamidines, N,N′-dihydroxyamidine esters, N,N′-dihydroxyamidine ethers and oxadiazolines results in them being initially efficiently absorbed after oral administration and subsequently being converted back by endogenous esterases and N-reduction into the actual active forms, the amidines (prodrug principle). The excellent absorbability of the modified amidine function in the gastrointestinal tract is apparently attributable to the greatly reduced basicity and the increased lipophilicity of the active ingredient molecules. The chemical modification of the amidine function to the N,N′-dihydroxyamidine function reduces the pKa of the amidine from about 11 to the about 4 of the N,N′-dihydroxyamidine and its ethers and esters. In the intestine, the main site of absorption of active ingredients, therefore, the N,N′-dihydroxyamidine or the N,N′-dihydroxyamidine ester and the N,N′-dihydroxyamidine ether are virtually completely in the form of the free base. In parallel with the decrease in the basicity through the modification made in the amidine function there is an increase in the lipophilicity of the corresponding active ingredients.

It is sufficient for the active ingredient to comprise at least one active amidine group in the proposed form. The active ingredient may accordingly comprise a plurality of amidine groups (e.g. two as in the case of pentamidine), in which case at least one of these groups is modified in the manner described above. It is equally possible to employ mixtures of active ingredients as long as at least one active ingredient has an amidine group. The oral dosage form can be prepared as liquid, semisolid or solid preparation, in particular as tablet, coated tablet, pellets or microcapsules. In this connection, for those embodiments in which liquid preparations are employed, the active ingredient or the mixture of active ingredients can be taken up in a suitable nontoxic solvent such as, for example, water, monohydric alcohols, especially ethanols, polyhydric alcohols, especially glycerol and/or propanediol, polyglycols, especially polyethylene glycols and/or miglyol, glycerol formal, dimethylisosorbitol, natural or synthetic oils. The customary bases are used to produce semisolid or solid preparations, such as, for example, bentonite, Veegum, guar gum and/or cellulose derivatives, especially methylcellulose and/or carboxymethylcellulose, and polymers of vinyl alcohols and/or vinylpyrrolidones, alginates, pectins, polyacrylates, solid and/or liquid polyethylene glycols, paraffins, fatty alcohols, petrolatum and/or waxes, fatty acids and/or fatty acid esters.

Solid preparations may further comprise extenders known per se, such as, for example, colloidal silica, talc, lactose, starch powder, sugar, gelatin, metal oxides and/or metal salts. Appropriate further additives are stabilizers, emulsifiers, dispersants and preservatives.

The medicinal substances modified by the use according to the invention exhibit excellent absorbability and thus bioavailability on oral administration, and thus the pharmacological effect of the amidine is distinctly increased. It is thus now possible to provide an optimal pharmaceutical form for oral use of amidines.

The use according to the invention is particularly important through the fact that the amidine functional group is an essential constituent of various important active ingredients for various areas of use. The amidine group is inter alia a constituent of the following active ingredient classes or active ingredients: protease inhibitors (thrombin inhibitors such as melagatran, inhibitors of factor Xa, factor VII and all proteases of the coagulation cascade; matriptase inhibitors), anticoagulants, thrombolytics, antifibrinolytics, DNA- and RNA-intercalating compounds (such as pentamidine, diminazene, isometamidium), N-methyl-D-aspartate receptor antagonists and inhibitors of viral enzymes (such as, for example, neuraminidase inhibitors).

Active ingredients which comprise an active amidine group can be employed inter alia for inhibiting the coagulation of blood, for the prophylaxis and therapy of visceral and cutaneous leishmaniosis, of trypanosomiasis (African sleeping sickness) of the pneumonia caused by Pneumocystis carinii (PcP), for inhibiting the growth of malignant tumors, lowering blood pressure, neuroprotection, and for controlling viral infections such as influenza and HIV infections.

The above lists are only by way of example, and the invention encompasses in principle all active ingredients which have at least one amidine group. The use according to the invention can thus be applied to a very wide range of active ingredient classes and indications and can distinctly increase the bioavailability of many medicinal substances whose active form comprises an amidine.

Examples which may be mentioned of medicinal substances modified according to the invention are N,N′-dihydroxybenzamidine and its derivatives according to the invention. N,N′-Dihydroxybenzamidine can be synthesized as described by Ley and Liu et al. [Ley H. (1898) Ber. Dtsch. Chem. Ges. 31 2126-2129; Liu K.-C.; Shelton B. R.; Hews R K. (1980) J. Org. Chem. 45 3916-3918]. Synthesis of its monoethers can follow the method of Ley et al. [Ley, H.: Ulrich, M. (1914) Ber. Dtsch. Chem. Ges. 47 2938-2944]. The diethers can be synthesized by O-methylation of the monoethers with, for example, methyl iodide. The mono- and diesters of N,N′-dihydroxybenzamidine are synthesized as described by Andrewes et al. [Andrewes, C. H.; King, H.; Walker, J. (1946) Proceedings of the Royal Society of London, Series B 133 20-62]. 4-Hydroxy-1,2,4-oxadiazoline can be synthesized as described by Desherces et al. [Desherces, S.; Barrans, J.; Roubaty, J. L. (1978) Revue Roumaine de Chimie 23 203-208].

To demonstrate the absorption from the gastrointestinal tract and the subsequent reduction to the free amidine, N,N′-dihydroxybenzamidine was chosen as model compound for the novel prodrug principle, and was administered orally and intravenously to three pigs. Metabolism of N,N′-dihydroxybenzamidine to benzamidine in vivo proceeds in the following way:

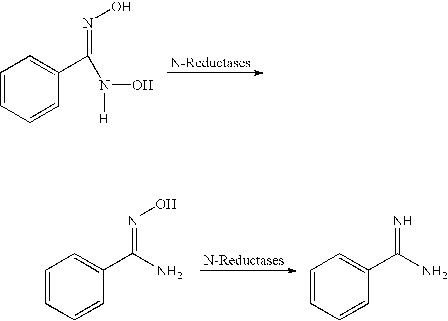

N,N′-dihydroxybenzamidine benzamide oxime benzamidine

In order to be able to ascertain the exact dosage of the substances, the animals were weighed once a week. The daily weight gain was calculated from the data. The substances to be administered orally were mixed into the moistened, ground feed concentrate. The substances given intravenously were dissolved in 0.9% NaCl solution in order to avoid hemolysis.

Directly before injection into the indwelling vein catheter, the solution was filtered in order to avoid induction of thrombus formation by any undissolved portions. The injection was followed by flushing with at least 10 ml of 0.9% NaCl solution again. The substance was administered in the morning on each occasion. A washout period took place the next day on each occasion in order to ensure complete excretion of the medicinal substance.

The orally administered doses of N,N′-dihydroxy-benzamidine were in each case 10 mg/kg of body weight (BW). The concentration of the substances administered intravenously as bolus was 2 mg/kg BW. Benzamidine and N,N′-dihydroxybenzamidine were likewise administered intravenously. The samples were taken at previously fixed times. The experimental period for one condition lasted one day. The blood samples were obtained over a period of 24 hours after administration of the substance. After oral administration, the samples were taken after 0, 30, 60, 90, 120, 150, 180, 240, 360, 480, 720 and 1440 minutes. After intravenous administration, an additional sample was taken after 5 and 15 minutes. The whole blood obtained was transferred into heparin tubes and centrifuged (4° C., 10 min, 1500 g). After centrifugation, about 4 ml of plasma were removed as supernatant, pipetted into Eppendorf vessels and frozen at −80° C. The plasma samples were slowly thawed and then centrifuged at 7000 rpm for 3 minutes, worked up by solid-phase extraction and passed on for HPLC.

The results of the experiments are depicted in the figures. These show:

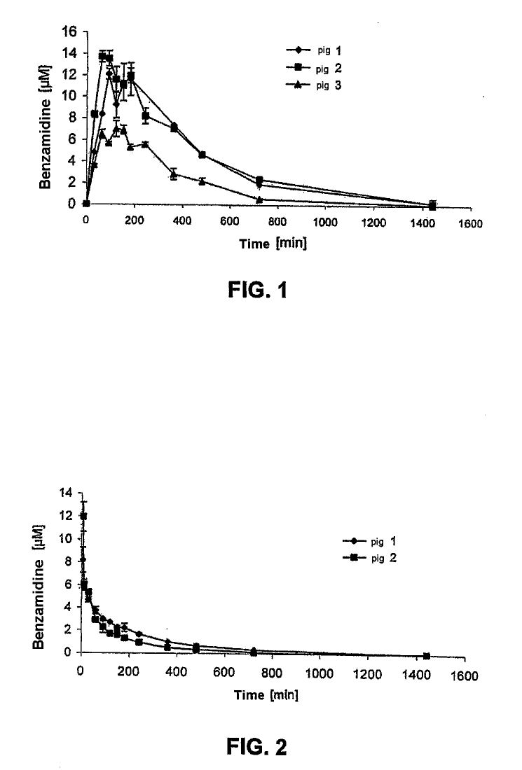

FIG. 1 the benzamidine plasma level plots after oral administration of N,N′-dihydroxybenzamidine (10 mg/kg BW) to three pigs,

FIG. 2 the benzamidine plasma level plots after injection (2 mg/kg BW) in two pigs,

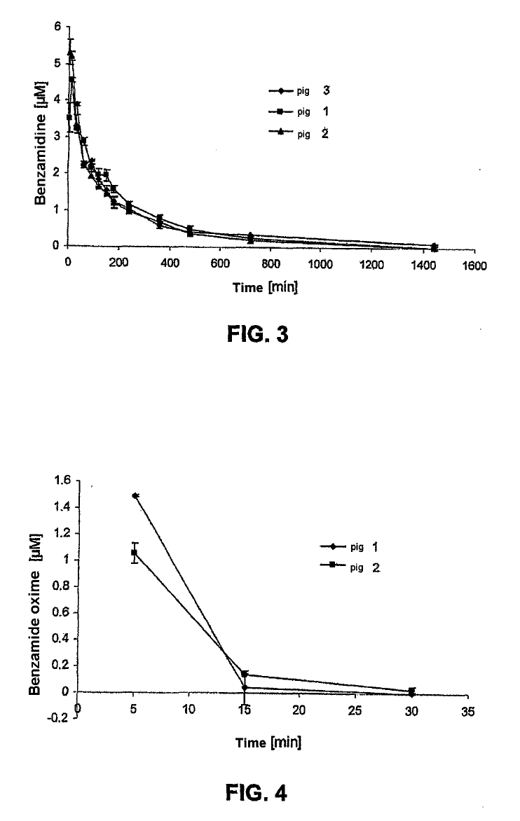

FIG. 3 the benzamidine plasma level plots after injection of N,N′-dihydroxybenzamidine (2 mg/kg BW) in three pigs, and

FIG. 4 the benzamide oxime plasma level plots after injection of N,N′-dihydroxybenzamidine (2 mg/kg BW) in two pigs.

It was possible to determine the oral bioavailability of benzamidine after oral administration of N,N′-dihydroxybenzamidine from the data obtained:

| | | | Standard |
|---|---|---|---|
| | Bioavailability | Mean | deviation |
| Animal | [%] | [%] | [%] |
| Animal 1 | 106.71 | | |
| Animal 2 | 113.90 | 90.62 | 34.28 |
| Animal 3 | 51.25 | | |

As is evident from the above table, benzamidine has a bioavailability of 90.62% after oral administration of N,N′-dihydroxybenzamidine. This shows that the prodrug is almost completely absorbed after oral administration and is rapidly reduced to the active form in the blood. After injection of N,N′-dihydroxybenzamidine too, the prodrug is rapidly reduced to the amidine, with benzamide oxime also being detectable in addition in the plasma after this mode of administration.

1. A method for improving the bioavailability of a medicinal substance that comprises an amidine functional group, which comprises replacing the amidine functional group in the medicinal substance by a functional group selected from the group consisting of N,N′-dihydroxyamidine (I), N,N′-dihydroxyamidine ether (II), N,N′-dihydroxyamidine diether (III), N,N′-dihydroxyamidine ester (IV), N,N′-dihydroxyamidine diester (V) and 4-hydroxy-1,2,4-oxadiazoline (VI) of the formulae 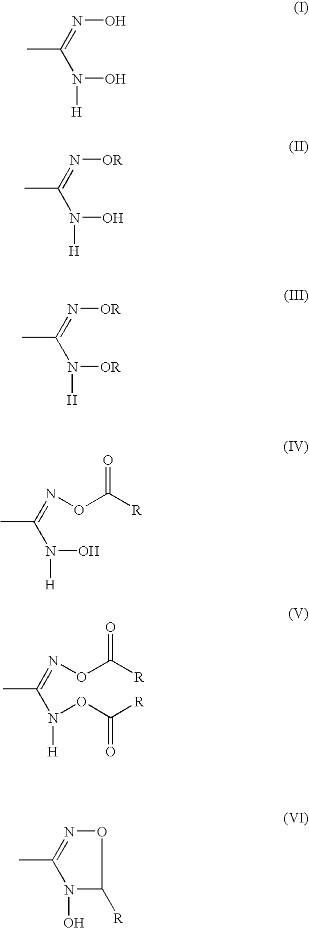
Where each R is independently selected from the group consisting of hydrogen, an alkyl radical and an aryl radical.

2. The method of claim 1, wherein the medicinal substance is selected from the group consisting of protease inhibitors, DNA- or RNA-intercalating compounds, inhibitors of viral enzymes, and N-methyl-D-aspartate receptor antagonists.

3. The method of claim 2, wherein the protease inhibitor is an inhibitor of a protease of the coagulation cascade.

4. The method of claim 2, wherein the protease inhibitor is a urokinase inhibitor.

5. The method of claim 2, wherein the DNA and RNA-intercalating compound is pentamidine, diminazene or isometamidium.

6. The method of claim 2, wherein the inhibitor of viral enzymes is a neuraminidase inhibitor.

7. The method of claim 2, wherein the medicinal substance is an N-methyl-D-aspartate receptor antagonist.

8. The method of claim 1, wherein the medicinal substance is employed for the prophylaxis and therapy of visceral and/or cutaneous leishmaniosis, trypanosomiasis or pneumonia caused by Pneumocystis carinii, for inhibiting the growth of malignant tumors, for inhibiting the coagulation of blood, for lowering blood pressure, for neuroprotection, or for fighting viral infections, including influenza and HIV infections.

9. The method of claim 2, wherein the protease inhibitor is a matriptase inhibitor.

10. The method of claim 3, wherein said inhibitor of a protease of the coagulation cascade is selected from the group consisting of a thrombin inhibitor, an inhibitor of factor Xa, and an inhibitor of factor VII.
